(19) United States
(12) Patent Application Publication
(10) Pub. No.: US 2022/0111378 A1
(43) Pub. Date: Apr. 14, 2022

(54) METHODS AND SYSTEMS FOR DETERMINING TARGET SENSITIVITY TO A THERAPEUTIC FORMULA

(71) Applicant: GENECAPTURE, INC., Huntsville, AL (US)

(72) Inventors: Paula M. KOELLE, Huntsville, AL (US); Krishnan CHITTUR, Huntsville, AL (US); Zachary MCGEE, Guntersville, AL (US); Thomas M. CANTEY, Huntsville, AL (US); Valentin KORMAN, Huntsville, AL (US); Greg THOMPSON, Scottsboro, AL (US)

(21) Appl. No.: 17/423,548

(22) PCT Filed: Jan. 17, 2020
(86) PCT No.: PCT/US2020/013996

§ 371 Date: Jul. 16, 2021

Related U.S. Application Data
(60) Provisional application No. 62/793,610, filed on Jan. 17, 2019.

(51) Int. Cl. B01L 3/00; C12Q 1/18; C12M 3/00; C12M 1/00

(52) U.S. Cl. CPC B01L 3/502; C12Q 1/18; C12M 23/42; C12M 43/00; G01N 2015/1006; B01L 2300/0681; B01L 2300/0645; B01L 2300/14; B01L 2300/021; B01L 2200/0689

(57) ABSTRACT

Systems and methods are disclosed herein for determining the sensitivity of a target to a therapeutic formula. The systems include a measurement device for measuring properties of a target sample fluid housed within a cartridge (the cartridge having at least one test compartment and at least one control compartment). Methods can include determining the concentration of the target suspended in a target sample fluid and placing the target sample fluid into at least one test compartment comprising a first therapeutic formula. The system, via a processor, measures the properties of the sample fluid, compares the measured properties to a threshold property, determines the sensitivity of a target species to a therapeutic formula, and presents an indicator of the target sensitivity to an end user. In some embodiments, the sensitivity data can be stored to a database for later use by an adaptive machine learning tool.

### RELATED APPLICATIONS

This application claims the benefit of priority to U.S. Provisional Application No. 62/793,610, filed Jan. 17, 2019, which is hereby incorporated by reference in its entirety and for all purposes.

### TECHNICAL FIELD

This application relates to the field of diagnostic testing, and more particularly, to testing for target sensitivity and resistance to therapeutic formulas.

### BACKGROUND OF THE INVENTION

In response to the growing problem of multi-drug resistant organisms (MDRO), new technologies are needed to rapidly identify the infectious agents causing disease and to determine their antimicrobial resistance (AMR) profile. Culture of the organism (phenotypic testing) is the current gold standard, and can take 24-48 hours for pathogen identification and another 1-2 days for AMR testing. Lab-based molecular tests for AMR profiling that rely on the amplification of known antimicrobial genes are becoming more widely available. However, such genotypic tests are expensive and can be potentially misleading, as they cannot identify all combinations of genes that may confer AMR and can identify the presence of an AMR gene even if it is not the specific mechanism of organism resistance. There are simply too many resistance mechanisms for a genotypic approach to be comprehensive (van Belkum, A. et al., Developmental roadmap for antimicrobial susceptibility testing systems. Nat Rev Microbiol 2019; 17:51).

The essential benefit of culturing the organism for drug screening is that the phenotypic resistance profile is determined—the organism experiences the actual effects of the drug and either grows or fails to grow in its presence. It is ultimately this phenotypic AMR profile that determines whether a particular drug will be effective in treating an infection or, through its ineffectiveness, allow the infection to continue.

Bacterial culture is currently the gold standard for antibiotic screening, but samples must typically be sent away from the point of care (a healthcare practitioner's office, for example) to a laboratory equipped to handle bacterial culture. The turnaround time from such a process can be 2-3 days. In the meantime, the patient may be started on an antibiotic that turns out to be the wrong choice because the organism is resistant. This trial and error process contributes to the rise of MDRO. The medical community would benefit greatly from simple, affordable, and quantifiable pathogen identification capable of being performed at the point of care, directly from patient samples, and followed by a rapid AMR determination to inform prescription decisions.

Resistances to therapeutic formulas can also occur in non-bacterial cells and/or pathogens. Cancer cells can evolve to be resistant to a standard chemotherapeutic regimen, for example. Fungal and viral infections can evolve to be resistant to antifungal and antiviral treatments. As such, the medical community would benefit not only from solutions for bacterial resistance, but for multiple other types of targets, including cancer cells, fungi, viruses, parasites, and other organisms.

### SUMMARY OF THE INVENTION

Systems and methods for determining target sensitivity to a therapeutic formula are disclosed herein. The disclosed systems and methods facilitate rapid, point of care testing to determine the sensitivity of a target infection to a range of treatment choices. This will improve the precision of treatment, thereby reducing the evolution of MDRO. The systems described herein are compact, portable, and facilitate use by medical staff of all experience levels. The methods described may be automated to save time and increase accessibility to clinics and hospitals. The sensitivity data can be shared to a database for later use by an adaptive machine learning tool. The adaptive machine learning tool can, among other things, pool data from multiple users to track the resistance of an organism to various treatments over the larger population, providing public health insights into geographic and longitudinal resistance trends.

The methods for determining the sensitivity of a target to a therapeutic formula include determining the concentration of the target suspended in a target sample fluid, placing at least a portion of the target sample fluid into at least one test compartment comprising a first therapeutic formula, measuring at least one measured property of the target sample fluid in the at least one test compartment under conditions favorable for target multiplication or growth, comparing the measured property to a threshold property, determining the sensitivity of the target to the first therapeutic formula based on a deviation of the measured property from the threshold property, and presenting an indicator of the sensitivity of the target to the first therapeutic formula. In some embodiments of the methods, the identity of the target is known.

In some embodiments, the at least one test compartment includes two or more test compartments. The method can further include placing varying therapeutic formulas into each of the two or more test compartments and determining the sensitivity of the target to each of the varying therapeutic formulas (based on a deviation of the measured property from a threshold property for each compartment of the two or more test compartments). Some embodiments of the method can include placing a portion of the target sample fluid into a control compartment, measuring a control property of the target sample fluid in the control compartment under conditions favorable for target multiplication or growth and in the absence of the at least one therapeutic formula, and using the measured control property in a calculation to determine the threshold property.

In some embodiments of the methods, the at least one measured property is an optical property, and measuring an optical property comprises measuring the optical density of the target sample fluid. The optical density can, in some embodiments, be measured at a wavelength between from 200 nanometers and 10,000 nanometers. In some embodiments, the optical density is measured at multiple wavelengths. In some embodiments, measuring the optical density includes measuring transmittance of a light beam over a path length of from 2 to 13 centimeters. The light beam can, in some embodiments, be reflected off at least one mirror or reflective compartment coating.

In some embodiments of the methods, the at least one measured property is an impedance property, and measuring the impedance property comprises moving the target sample fluid within a test compartment past at least one electrode. In some embodiments, the target sample fluid can be moved repeatedly between a first lobe of the test compartment and a second lobe of the test compartment using, for example, pneumatic pressure. In some embodiments, an impedance flow cytometry analysis is performed on the target sample fluid.

In some embodiments, the target sample fluid comprises a metabolite or a nutrient (such as, but not limited to glucose). The at least one measured property is a change in a concentration of the nutrient or metabolite. Some embodiments of the methods include measuring a change in the electrical charge of the target sample fluid (for example, if the target sample fluid includes charged nanoparticles).

Measuring at least one measured property of the target sample fluid can further include taking multiple measurements of the measured property over a time period. In some embodiments, the sensitivity of the target to the first therapeutic formula can be determined within a time period of from 5 minutes to 360 minutes. The multiple measurements of the measured property over a time period can be used to construct at least one test growth curve and at least one control growth curve. Comparing the measured property to a threshold property further comprises comparing the test growth curve to the control growth curve.

Assay results including the data on the sensitivity of the target to the first therapeutic formula can be stored to the memory of an adaptive machine learning tool, and the assay results can be used by the adaptive machine learning tool in longitudinal and/or geographic analyses of target sensitivities and resistances to particular therapeutic formulas. In some embodiments of the methods, the assay results can be accessed by the adaptive machine learning tool to predict target sensitivities and resistances to therapeutic formulas or candidates. In some embodiments, the adaptive machine learning tool can pool target sensitivity results and use the pooled target sensitivity results to predict epidemics, pandemics, or early stage outbreaks.

In some embodiments of the methods, flow of the target sample fluid into the at least one test compartment is automated. The target sample fluid can be prepared by automated preparation steps prior to placement in the at least one test compartment, or it can be prepared by manual preparation steps prior to placement in the at least one compartment.

Systems for determining the sensitivity of a target to a therapeutic formula are disclosed herein. The systems include a measurement device configured to measure properties of a target sample fluid housed within a cartridge. The cartridge includes at least one test compartment and at least one control compartment. The system further includes a processor in communication with the measurement device and a memory. The processor executes computer readable instructions stored on the memory, which cause the processor to perform the steps of: measuring a measured property of the target sample fluid in the at least one test compartment under conditions favorable for target multiplication or growth, comparing the measured property to a threshold property, determining the sensitivity of the target to a first therapeutic formula based on a deviation of the measured optical property from the threshold property, and presenting an indicator of the sensitivity of the target to the first therapeutic formula.

The cartridge can include two or more test compartments. In some embodiments, the cartridge is relatively small (for example, less than or equal to 8 inches in width, less than or equal to 10 inches in height, less than two pounds in weight, and/or from about 2 ounces to about 6 ounces in weight). The cartridge can include, or can be formed of, a plastic material and can include one or more directional indicators. In some embodiments, the cartridge does not require cold storage.

In some embodiments, the cartridge includes a filtering chamber. The filtering chamber can be fluidically coupled to a target sample fluid input well and at least one test compartment. The filtering chamber can be fluidically coupled to a mixing well that is positioned between the filtering chamber and the at least one test compartment.

In some embodiments, the measurement device of the system is an impedance flow cytometry device including a voltage source and an amplifier. The at least one test compartment is associated with at least one electrode pair for use during impedance flow cytometry. In some embodiments, the at least one test compartment includes multiple lobes bridged by at least one channel, and the electrode pair is positioned adjacent to the at least one channel. In some embodiments, the test compartment is associated with a first electrode pair positioned near the top of the channel and a second electrode pair positioned near the bottom of the channel, wherein a positive electrode of the first electrode pair is adjacent to a first lateral side of the channel, and a positive electrode of the second electrode pair is adjacent to the opposite lateral side of the channel.

In some embodiments, the measurement device of the system is an optical property measurement device. The optical property measurement device includes a light source for emitting a light beam, a detector for detecting a light beam, and a path length across which the light beam travels. The at least one test compartment can be elongate and configured to act as a light guide channel. In some embodiments, the path length can be from about 2 centimeters to about 13 centimeters. Some embodiments can include at least one object that extends the path length beyond the physical distance between the light source and the detector such that the path length is greater than the distance between the light source and the detector.

In some embodiments, the cartridge is fluidically sealed and houses growth media and at least one therapeutic formula. The system can further include a cutting mechanism in communication with the processor. The processor can instruct the cutting mechanism to break a fluidic seal of the cartridge. Some embodiments can include a cartridge movement mechanism in communication with the processor.

Some embodiments can include an automated sample preparation system in communication with the processor. Some embodiments can include a fluidic and pressure handling system that is configured to couple to fluidics and pressure ports on the cartridge. Some embodiments can include at least one environmental control mechanism in communication with the processor. The measurement device and the environmental control mechanisms can be at least partially enclosed in a housing.

In some embodiments, the system for determining the sensitivity of a target to a therapeutic formula is configured to receive inputs regarding the identity of the target, either manually, or from a target identification system, or both.

### BRIEF DESCRIPTION OF THE DRAWINGS

The invention is explained in even greater detail in the following drawings. The drawings are merely examples to illustrate the structure of preferred devices and certain features that may be used singularly or in combination with other features. The drawings are not necessarily to scale. The invention should not be limited to the examples shown. In the drawings, like reference numbers and designations in the various drawings indicate like elements.

FIG. 1A is a view of a benchtop set up including a target sensitivity testing module and a computer.

FIG. 1B is a top-down view of a cross-section taken through line 1B-1B of FIG. 1A.

FIG. 2A is a view of a benchtop setup of another target sensitivity testing module.

FIG. 2B is an enlarged view of the optical property measurement device of the target sensitivity testing module shown in FIG. 2A.

FIG. 3A is a top down view of a channel of an impedance flow cytometry (IFC) measurement device that can be used with a target sensitivity testing system.

FIG. 3B is a graph of an impedance trace from an IFC measurement device such as the one shown in FIG. 3A.

FIG. 4 shows an example of a cartridge that can be used with an IFC measurement device.

FIG. 5 shows an example compartment from an IFC cartridge such as the one shown in FIG. 4.

FIG. 6 shows an example of a cartridge that can be used with an optical measurement device.

FIG. 7 is a flow chart representing an example method for determining target sensitivity to a therapeutic formula.

FIG. 8 is a view of another benchtop setup of a target sensitivity testing module. In the embodiment of FIG. 8, a target identification module is also included and is in communication with the target sensitivity testing module.

FIG. 9 shows simultaneously collected growth curves of a clinical isolate of K. pneumoniae KpH201 when exposed to breakpoint concentrations of ampicillin, tetracycline, and ciprofloxacin. Culture results for this strain determined sensitivity to ampicillin and resistance to both tetracycline and ciprofloxacin. These results are confirmed within approximately 20 minutes by comparing each curve to the control containing no antibiotic.

FIG. 10 shows simultaneously collected growth curves of a clinical isolate of Enterococcus faecalis EfR100 when exposed to breakpoint concentrations of ampicillin and ciprofloxacin. Culture results for this strain determined sensitivity to both ampicillin and ciprofloxacin. These results are confirmed within approximately 33 minutes by comparing each curve to the control containing no antibiotic. Each growth curve is fitted with a 3rd order polynomial to mitigate noise interference as denoted by dashed lines.

FIG. 11 shows simultaneously collected growth curves of a clinical isolate Staphylococcus aureus SaR100 when exposed to breakpoint concentrations of ampicillin, tetracycline, and ciprofloxacin. Culture results for this strain determined sensitivity to ampicillin, tetracycline, and ciprofloxacin. These results are confirmed within approximately 42 minutes by comparing each curve to the control containing no antibiotic. Each growth curve is fitted with a 3rd order polynomial to mitigate noise interference as denoted by dashed lines.

FIG. 12 shows simultaneously collected growth curves of a clinical isolate of Pseudomonas aeruginosa PaH201 when exposed to breakpoint concentrations of ampicillin, tetracycline, and ciprofloxacin. Culture results for this strain determined sensitivity to ciprofloxacin and resistance to ampicillin and tetracycline. These results are confirmed for ampicillin and ciprofloxacin within approximately 42 minutes by comparing each curve to the control containing no antibiotic. Each growth curve is fitted with a 3rd order polynomial to mitigate noise interference as denoted by dashed lines.

FIG. 13 shows simultaneously collected growth curves of a clinical isolate of Escherichia coli EcUAH202 when exposed to breakpoint concentrations of ampicillin, tetracycline, and levofloxacin. Culture results for this strain determined sensitivity to levofloxacin and resistance to ampicillin and tetracycline. Confirmation of these results for ampicillin and levofloxacin was possible within approximately 7 minutes by comparing each curve to the control containing no antibiotic.

FIG. 14 shows simultaneously collected growth curves of a sterile urine sample spiked with clinical isolate EcUAH202 when exposed to breakpoint concentrations of ampicillin, tetracycline, and ciprofloxacin. Culture results for this strain determined sensitivity to ciprofloxacin and resistance to ampicillin and tetracycline. Confirmation of this results for ampicillin was possible within approximately 5 minutes by comparing each curve to the control containing no antibiotic.

FIG. 15 shows simultaneously collected growth curves of a clinical isolate of Candida tropicalis CanTCDC345 when exposed to breakpoint concentrations of fluconazole and caspofungin. Culture results for this strain determined sensitivity to caspofungin and resistance to fluconazole. Confirmation of this results for caspofungin was possible within approximately 12 minutes by comparing each curve to the control containing no antimycotic. Each growth curve is fitted with a 3rd order polynomial (dashed line) to mitigate noise interference. Y-axis values are fold increase in optical density in relation to each well's initial time point.

FIG. 16 shows a graph with results of an antibiotic susceptibility testing (AST) assay on K. pneumoniae KpH201, taken at 5 second intervals for 37 minutes, using the methods disclosed herein and the system shown in FIGS. 2A and 2B. The data show that the KpH201 strain is sensitive to CIP.

FIG. 17 is a graph showing the “time to result” or “time to call” for six different types of bacteria. Antibiotic susceptibility is determined in 5 minutes to 75 minutes.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed.

Throughout this application, various publications and patent applications are referenced. The disclosures of these publications in their entireties are hereby incorporated by reference into this application in order to more fully describe the state of the art to which this disclosure pertains. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

As used herein, “target” or “target species” can refer to a bacterium, a cell (such as a cancer cell), a microorganism, fungus, parasite, virus or pathogen. In an aspect, the target can be sourced from a sample, such as an environmental sample, a biological sample, an animal sample, or a human sample. A “known” target is a target that has been identified. For example, a “known” target is a target that has been identified prior to initiating the methods for determining the sensitivity of the target to a therapeutic formula. A “therapeutic formula” can include a single therapeutic candidate, or it can include multiple therapeutic candidates. It can refer to a chemotherapeutic or therapeutic compound or molecule, such as an anticancer drug, an antibiotic, or an antimycotic, antifungal or antiparasitic drug. In some embodiments, a therapeutic formula can limit the growth or proliferation of a target (for example, if the target is susceptible or sensitive to the therapeutic formula). Varying therapeutic formulas can have the same therapeutic candidate in varying amounts or concentrations, or multiple therapeutic candidates in varying amounts or concentrations. The term formula can indicate that the candidate is in a suspension or solution with a fluid, or it can indicate that the candidate is in a dry form.

A “threshold” as it pertains to “threshold property” is a number or numerical range that can be compared to a measured property to indicate whether growth of a target is significant and therefore, if the target is sensitive to, or susceptible to, a therapeutic formula. For example, the threshold number can relate to the level of growth of a target in the absence of a therapeutic formula (for comparison against growth of the same target in the presence of a therapeutic formula). For example, optical transmittance relates to the turbidity of a solution, and solutions with higher concentrations of target species are more turbid. A high transmittance indicates a clear solution and a low transmittance indicates a turbid solution. Thus, measured transmittance above a threshold transmittance can indicate a clear solution, and insignificant target growth, whereas measured transmittance around or below a threshold transmittance can indicate turbidity, which indicates significant target growth. Conversely, optical density increases as a target solution becomes more turbid. Therefore, measured optical density around or above a threshold optical density can indicate a turbid solution, or significant bacterial growth. As another example, the impedance of a solution increases with cellular proliferation. Therefore, an impedance measurement around or above a threshold impedance can indicate significant target growth, whereas an impedance measurement below a threshold impedance can indicate insignificant target growth.

Two components said to be “fluidically coupled” can be directly contacting one another, or they can be separated from each other (spaced by other components), so long as a fluid path exists between the two fluidically coupled components.

The methods and systems disclosed herein describe a robust, enzyme-free assay for rapid and direct identification of bacterial, viral and fungal pathogens (target species) direct from a patient sample with a follow-on phenotypic AMR test in as little as 5 minutes. With simple, easy to use cartridges to identify up to dozens of pathogens per sample and with the ability to test therapeutic options at once, it is possible to provide sample-in/answer-out, affordable, point of care (POC) testing in a variety of medical facilities including but not limited to healthcare practitioner offices, emergency rooms, urgent care centers, pharmacy clinics, and in the field (for example, disaster zones, conflict zones, refugee camps, outbreak zones, and/or remote areas with limited access to a centralized healthcare system). Rapidly facilitating effective treatment decisions will greatly improve patient outcomes. As healthcare decentralizes to the POC, a straightforward value-proven platform assay that allows immediate, targeted treatment will keep costs down. The methods disclosed can also provide important information specific to antibiotic stewardship (i.e., helping clinics and hospitals to avoid overuse of antibiotics and to avoid contamination).

International application no. PCT/US2017/037806 (Publication No. WO 2017/218858) is jointly owned and is incorporated by reference herein in its entirety. WO 2017/218858 discloses the “CAPTURE” (Confirm Active Pathogens Through Unamplified RNA Expression) bench-top pathogen identification assay with automated hybridization and imaging components. The CAPTURE assay provides correct identification of over a dozen pathogens and also provides concentration readings. Once a pathogen is identified in sufficient quantity to be called an infection, the target sensitivity determination system disclosed herein (a rapid AMR assay) can be initiated at the point of care from a remaining aliquot of the biological sample or a fresh sample as needed. The target sensitivity determination system is used to rapidly determine the effectiveness of various therapeutic formulas against the pathogen, and can provide AMR discrimination in as little as 5-360 minutes.

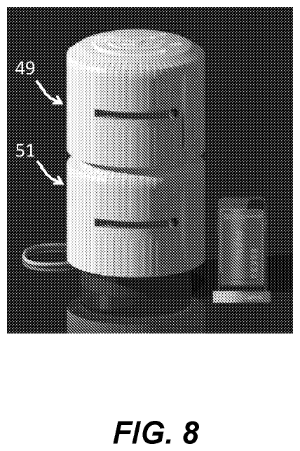

In some embodiments, a bench-top product can have both the CAPTURE identification module and the target sensitivity determination system module stacked in a modular configuration for ease of use and to limit footprint (such as is shown in FIG. 8). And, in some embodiments, the target sensitivity determination system module can be incorporated into the same device as the CAPTURE module. The target sensitivity determination system can be housed in a separate cartridge from the CAPTURE assay, with the pre-dispensed therapeutic formulas on board. That target sensitivity determination system cartridge can be loaded into the bench-top product in a similar manner as the CAPTURE cartridge, and discarded into the hazardous waste when the assay is complete.

While it is envisioned that the CAPTURE and the target sensitivity determination system assays can be used in conjunction with each other, it is also understood that the target sensitivity determination assay can be performed on its own, without input from the CAPTURE assay. And, while the systems and methods disclosed herein are described in the context of bacterial infections, it is understood that the inventive concepts can be applied to the treatment of other types of pathogenic infections (bacterial, viral, fungal, parasitic, for example) as well as to the testing of chemotherapeutic treatments.

Competing technologies that also test for target sensitivity and resistance are procedurally complex, computationally complex, large, and/or not easily portable. Some conventional technology in this space is designed to be stationary and housed in a laboratory environment (where it could be surrounded by supporting laboratory instruments). Other approaches are procedurally and computationally complex. For example, some technologies interpret images of bacteria on a surface, monitoring bacterial movement to determine sensitivity or resistance. This approach requires bacteria to be plated onto the surface and resulting images to be processed via software. By comparison, the systems and methods disclosed herein directly assess multiplication of the target over time from the original biological sample with little to no need for manual sample preparation. The device is portable and relatively small by comparison to conventional AST technologies, such that it can be more easily used at the point of care.

### Systems for Determining the Sensitivity of a Target to a Therapeutic Formula

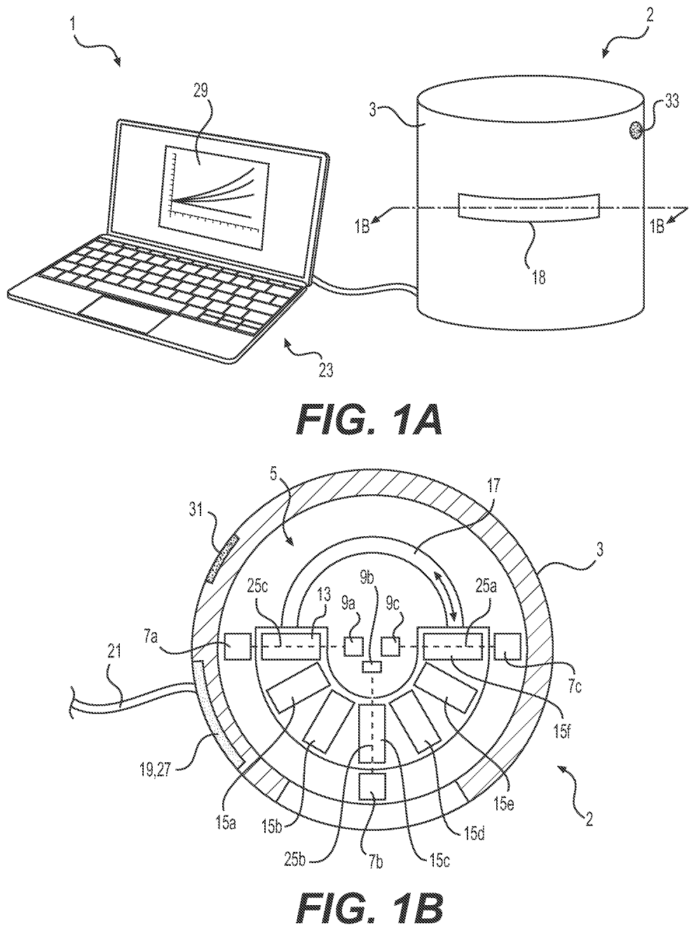

An example system for determining the sensitivity of a target to a therapeutic formula (a target sensitivity determination system) is shown in FIGS. 1A and 1B. The system shown in FIGS. 1A and 1B includes an optical measurement device, but other systems can include other types of measurement devices, such as IFC measurement devices, as will be described in greater detail, below. FIG. 1A shows a side view of the system 1 (not drawn to scale) and FIG. 1B shows a top-down view of a cross-section taken through line 1B-1B of FIG. 1A. The example system 1 includes a sensitivity testing module 2 comprising housing 3, which inside includes an optical property measurement device 5. Three light sources 7a, 7b, and 7c are positioned at intervals around the interior circumference of housing 3. These light sources may, in some embodiments, have different wavelengths. In some embodiments, the light sources are lasers, LEDs, or a combination thereof. The wavelength or wavelengths of the light sources are chosen to both mitigate optical interference from the growth medium and therapeutic formula and to optimize scattering, thus increasing resolution and reducing effective “time to call.” Advantageously, light sources with varying wavelengths can be provided to ensure that growth can be tracked regardless of any interference that may be caused by the therapeutic formula or growth media components at a single specific wavelength. Three detectors 9a, 9b, and 9c are positioned near the center of the sensitivity testing module 2. A cartridge 11 is positioned within the housing 3 such that compartments within the cartridge 11 are positioned between the light sources 7a, 7b, 7c and the detectors 9a, 9b, 9c. The system shown is exemplary only, and is not meant to limit the scope of the invention. For example, other embodiments may have more or fewer light sources, more or fewer detectors, differently shaped cartridges, or differently shaped housings.

The example cartridge 11 shown in FIG. 1B includes a control compartment 13 and a plurality of test compartments 15a, 15b, 15c, 15d, 15e, and 15f for housing therapeutic formulas to be tested against the pathogen or target. The system can include a fluidics and pressure handling system that couples to ports on the cartridge to facilitate the movement of the fluid therethrough. The number of test compartments is variable and is not meant to be a limiting factor. High throughput embodiments are envisioned with tens and even hundreds of test compartments, as well as multiple control compartments. Cartridge 11 is inserted into housing 3 via slot 18, for example, and may be provided with visual directional indicators that facilitate proper insertion (printed arrows, labels, or a shape that promotes proper insertion, for example).

Other features of the cartridge, including a small physical size, tear open packaging, long storage life, and simple instructions, may impart ease of use by point of care staff of all levels. The cartridge may be easily transported, stored, and disposed of in standard hazardous waste disposal containers, being formed of materials that do not require specialized environmental handling or controls (such as cold storage, for example). For example, the cartridge 11 can be formed of molded plastic that enables a closed system for fluid flow into the compartments. The material is transparent to facilitate the measurement of optical properties. In some embodiments, the cartridge can be less than 8 inches in width, and less than 10 inches in height. In some embodiments, the cartridge can weigh less than 2 pounds (for example, the weight can range from about 2 ounces to about 6 ounces).

In some embodiments, the cartridge may be provided to the end user with a control formula (such as bacterial growth medium) pre-loaded into the control compartment 13 and therapeutic formulas pre-loaded with growth medium into the test compartments and 15a-f (such as, for example, bacterial growth medium comprising a variety of antibiotic options). Alternatively, the cartridge may be provided to the end user with the therapeutic formulas alone (in dried or liquid state), and the growth medium added at the point of care. Or, alternatively, all compartments could come pre-loaded with growth medium alone, and the therapeutic formula options added at the point of care.

In FIG. 1B, an example semi-circular cartridge 11 is shown positioned within the sensitivity testing module 2 on a cartridge movement mechanism (rotating platform 17). The shape of the cartridge and positioning of cartridge components is exemplary only and is not meant to be limiting. A processor 19 controls movement of the platform 17 and activity of the light sources 7a, 7b, 7c. The processor 19 receives data from detectors 9a, 9b and 9c, which is then transmitted (wirelessly or via cord 21) to a device with a user interface 29, such as a desktop computer or the laptop 23 shown in FIG. 1A.

Path lengths 25a, 25b, and 25c extend between light sources 7a, 7b, 7c and detectors 9a, 9b, and 9c. In some embodiments, the path lengths can range from about 2 to about 13 centimeters. The path length, or the total distance that the light travels before detection, is relatively long by comparison to conventional optical property measurement systems. This relatively long path length ensures that even small changes in pathogen/target multiplication are detected. In the embodiment shown in FIG. 1B, the path length is the physical distance between the light source and the detector. However, various objects, such as, for example, mirrors and fibers, can be positioned within sensitivity testing module 2 to extend the effective path length beyond the physical distance between the light sources 7a, 7b, 7c and the detectors 9a, 9b, 9c. In some embodiments, for example, a detector and light source may be positioned on the same side as the sample compartment, and a mirror positioned at the other side of the sample compartment, to double the path length the light travels before detection. In some embodiments, the sides of a shaped or twisted sample compartment can be mirrored so that the light does not go down the middle, but is reflected from side to side as it travels through a sample compartment, thus increasing the effective path length. As such, the shape of the compartments shown in FIGS. 1A and 1B are not meant to be limiting. In another embodiment, the sides of a compartment may be fully or partially surrounded, or coated, by a reflective material such that the light beam reflects directly off the sides of the compartment at many points to extend the effective path length.

As mentioned above, processor 19 is in communication with the cartridge movement mechanism 17, the light sources 7a, 7b, 7c, and the detectors 9a, 9b, and 9c of the sensitivity testing module 2. The processor is also in communication with a memory 27, and executes computer readable instructions stored on the memory 27 to control various subsystems of the sensitivity testing module 2. The instructions cause the processor 19 to measure one or more optical properties, such as optical transmittance, of a test sample fluid that includes the pathogen/target and a therapeutic formula (for example, the target sample fluid in the at least one test compartment 15a) using detectors 9a, 9b, 9c. The instructions may also cause the processor 19 to compare the measured optical property to a threshold optical property (such as an optical transmittance of the control sample fluid in the control compartment 13, in the absence of a therapeutic formula). The instructions may further cause the processor 19 to determine the sensitivity of the target to the first therapeutic formula based on a deviation of the measured optical property from the threshold optical property, and to present an indicator of the sensitivity of the target to the first therapeutic formula. The indicator can be, for example, a graph on the user interface 29 of a laptop 23 connected by cord 21 to the processor 19. The processor 19 may be positioned within housing 3 or may be positioned external to the sensitivity testing module 2. In some embodiments, some processing may occur within an external software program, such as one housed on laptop 23, such that the on-board processor 19 serves to collect raw optical data and export it to the external software for calculations, including the comparison of the measured optical property to the threshold optical property and subsequent sensitivity determination. As used herein, the term processor is meant to include such external software.

The processor 19 can also be in communication with one or more environmental control mechanisms 31, which are responsible for controlling the environmental conditions within housing 3 to allow for multiplication of the target or pathogen. For example, the environmental control mechanism 31 can include temperature sensors, heaters and/or cooling elements to control the temperature within the housing 3, vibrational elements to shake the platform 17 for optimal target growth, and/or gas sensors and controls to optimize carbon dioxide and oxygen levels within the housing. Gas concentrations can be controlled by either mechanical or chemical mechanisms. The sensors associated with the environmental control mechanism 31 can relay information to the processor 19, and the processor 19 then executes instructions stored on the memory to control the environment in a certain way.

The processor and memory 19, 27 may also be equipped to control an automated sample preparation and/or fluidic handling system. The automated sample preparation can take place within housing 3 of the sensitivity testing module 2, or via an external sample preparation module that is in fluidic communication with the sensitivity testing module 2. The raw or prepared sample may be inserted into the housing 3, for example, at sample inlet port 33 (shown in FIG. 1A). The processor can control an automated fluid handling system of fluidic channels, pumps, vacuum, gravity feeds, and valves that lead the sample to fill compartments of the cartridge 11. The processor may also control a cartridge cutting mechanism that breaks the fluidic seal of the cartridge such that the sample can be loaded to the control and test compartments 13 and 15a-f. The power supply to both the sensitivity testing module 2 and the sample preparation module can be wired or wireless (batteries, for example).

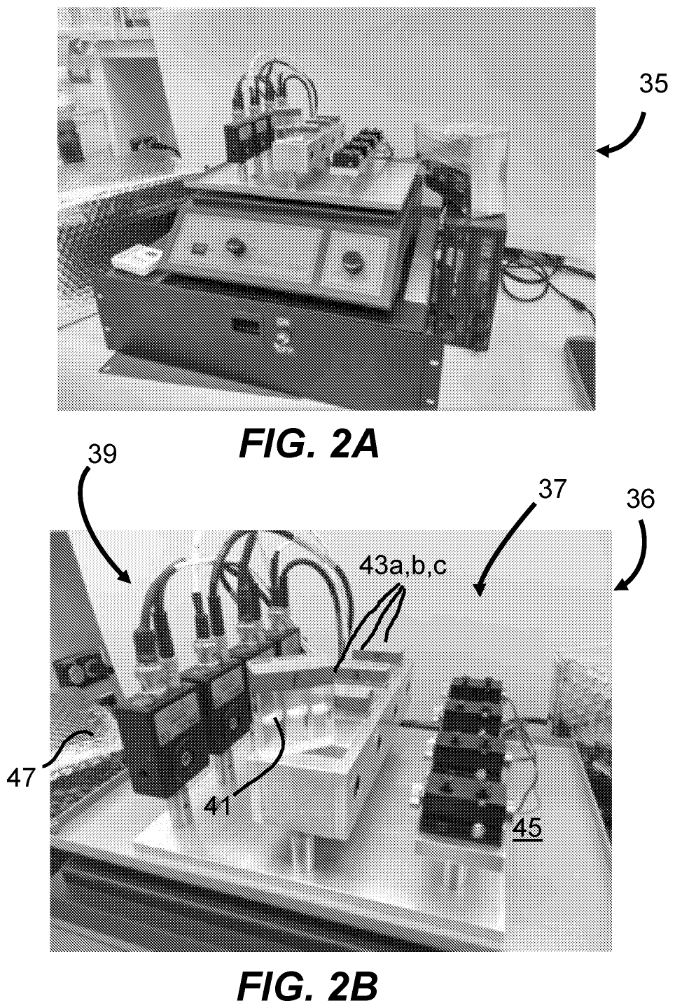

An alternative embodiment 35 of a target sensitivity testing determination system is shown in FIG. 2A and magnified in FIG. 2B. An optical property measurement device 36 includes a set of light sources 37 that send light through a control compartment 41 that houses a control sample in the absence of therapeutic formula and the test compartments 43a, 43b, and 43c housing test samples mixed with one or more therapeutic formulas. The optical properties of the light are detected by set of detectors 39 on the other side of the control and test compartments. Platform 45 functions as a movement mechanism to vibrate the sample for optimal target multiplication conditions. A foil cover 47 is provided as a housing. The foil cover 47 can be placed over the optical property measurement device 36 to allow for better maintenance of temperature and gas concentrations, as well as to provide a dark environment to optimize detection of optical properties.

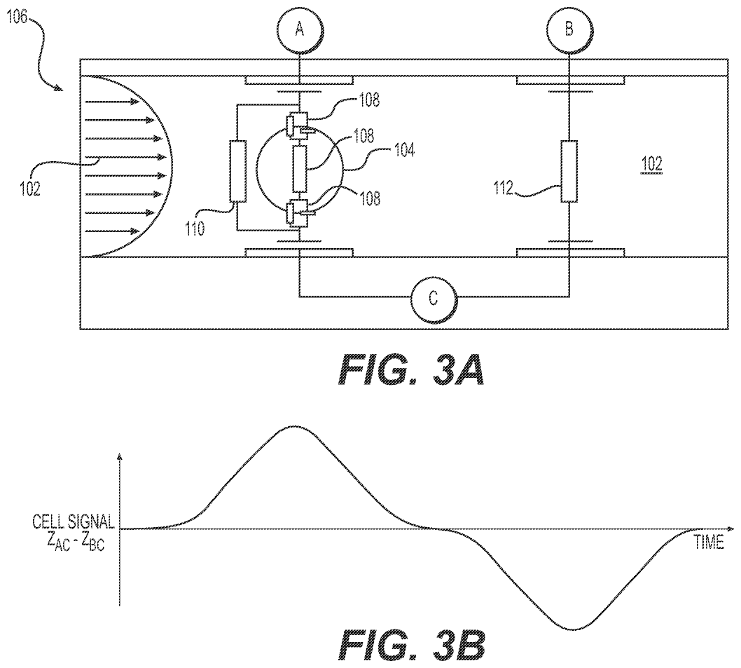

In another embodiment, the target sensitivity determination system disclosed herein can include an impedance flow cytometry (IFC) measurement device. A portion of an IFC measurement device is shown in FIG. 3A. In such a device, the target sample fluid 102 includes a weak electrolyte (generally, target growth media, such as cell or bacterial media, can act as a weak electrolyte). The target sample fluid, which contains target species 104 (such as, but not limited to, a bacterial cell), is passed through a small aperture or channel 106. The channel is long enough to allow passing species to achieve a uniform flow. Scientific concepts behind impedance flow cytometry are at least partially described in U.S. Pat. No. 10,048,190, which is hereby incorporated by reference in its entirety for teaching purposes only (i.e., this disclosure is not intended to be limited to concepts described in U.S. Pat. No. 10,048,190). Briefly, a target species 104 is associated with its own electrical properties 108 (membrane capacitance, cytoplasmic resistance, for example). The target electrical properties 108 are depicted as an electrical diagram in FIG. 3A. The target species electrical properties 108 differ from those of the target sample fluid electrical properties 110, 112 (resistance of the fluid, for example). Using excitation electrodes A, B and ground electrode C, the changes in impedance when a target species 104 passes through the channel 106 can be detected as shown in FIG. 3B. This detection allows for measurement and quantification of particles in the sample and can be used to note a change in the number of target species 104. By measuring the impedance in each of the test chambers of a cartridge, including the control chambers without a therapeutic formula, target growth curves can be determined that give information about the effectiveness of one or more therapeutic formulas. Computer analysis of impedance changes as particles pass through the tube allows the discrimination of particles of various shapes and sizes; and can measure the increase in particles in the flow, correlating to greater or lesser bacterial microorganism counts.

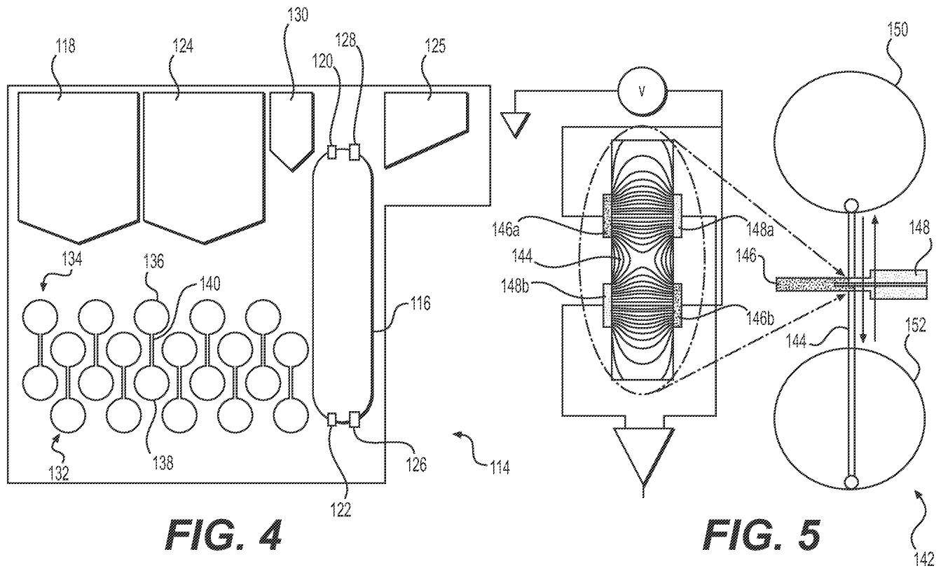

An IFC device includes a voltage source and an amplifier. An IFC cartridge can be provided that couples with the voltage source and the signal amplifier. The cartridge can couple to the fluidics and pressure handling system of the larger system via ports on the cartridge. Valves, fluidic lines, and pressure lines are included in the cartridge to assist with the flow of fluid therethrough. An example IFC cartridge 114 is shown in FIG. 4, but the positioning of all IFC cartridge components can vary. As such, some fluidic and pressure valves, fluidic and pressure lines, and electrical contacts are not shown in FIG. 4.

The IFC cartridge 114 can include a filtering chamber 116 having a filter. The IFC cartridge 114 can also include a target sample fluid input well 118 fluidically coupled to a target sample fluid inlet 120, which leads the target sample fluid into the filtering chamber 116. A waste outlet 122 is fluidically coupled to the filtering chamber 116 on the opposite side of the filter, such that target species from the input well gets caught in the filter and the remainder of the fluid travels to waste well 124. The filtering chamber 116 can also, in some embodiments, be fluidically coupled to backflush chamber 125, a backflush inlet 126 and a backflush outlet 128 (with the filter of the filtering chamber 116 positioned between backflush inlet 126 and backflush outlet 128). Backflush inlet 126 can be fluidically coupled to 125. In some embodiments, backflush outlet 128 is fluidically coupled to a mixing well 130. In this way, fluid from backflush chamber 125 can rinse trapped target species out of the filtering chamber 116 and into mixing well 130.

The mixing well 130 can include fluidic couplings to the larger system for providing additional target growth media to adjust the concentration of target species. The mixing well 130 can also be fluidically coupled to one or more test compartments 132, which can include preloaded therapeutic formulas in some embodiments. The cartridge 114 can also include one or more control compartments 134 that do not receive a target species and/or do not include a therapeutic formula. In some embodiments, backflush outlet 128 can be fluidically coupled directly to the test and/or control compartments 132, 134.

For an IFC cartridge, the test compartments can be associated with IFC electrodes. Flow cytometry variations of the Coulter method allow electrodes to be placed around or adjacent to a small channel connecting the two compartments. In some embodiments, the compartments 132, 134 can be configured with multiple lobes. For example, the compartments of FIG. 4 have two lobes 136, 138. The lobes can be, for example, approximately 8 millimeters in diameter and 0.5 millimeters in depth, and hold a volume of approximately 100 microliters of fluid. However, the size of the lobes can vary. The two lobes are fluidically coupled by a channel 140 extending therebetween. Electrode positioning is shown in FIG. 5. FIG. 5 shows a top view of an exemplary bi-lobed compartment 142 on the right, and a side view of a channel 144 on the left (looking along the longitudinal axis of said channel). In the embodiment shown, the IFC electrodes 146, 148 are positioned adjacent to the channel 144 to take IFC measurements as target sample fluid is passed back and forth between the lobes 150, 152 (using the aforementioned fluid and pressure handling system, ports, and valves). In the embodiment shown in FIG. 5, two pairs of IFC electrodes are provided: a first, upper pair 146a, 148a and a second, lower pair 146b, 148b. The upper pair is positioned near the top of the channel 144, while the lower pair is positioned near the bottom of the channel 144. The lateral configuration of the positive electrode 146a and negative electrodes 148a of the top pair is opposite the lateral configuration of the positive electrode 146b and negative electrode 148b of the lower pair. Said another way, the positive electrode 146a of the first electrode pair positioned near the top (upper) portion of the channel 144 is adjacent a first lateral side of the channel, and the positive electrode 146b of the second electrode pair (positioned near the bottom or lower portion of the channel), is adjacent the opposite lateral side of the channel. This configuration may improve signal detection.

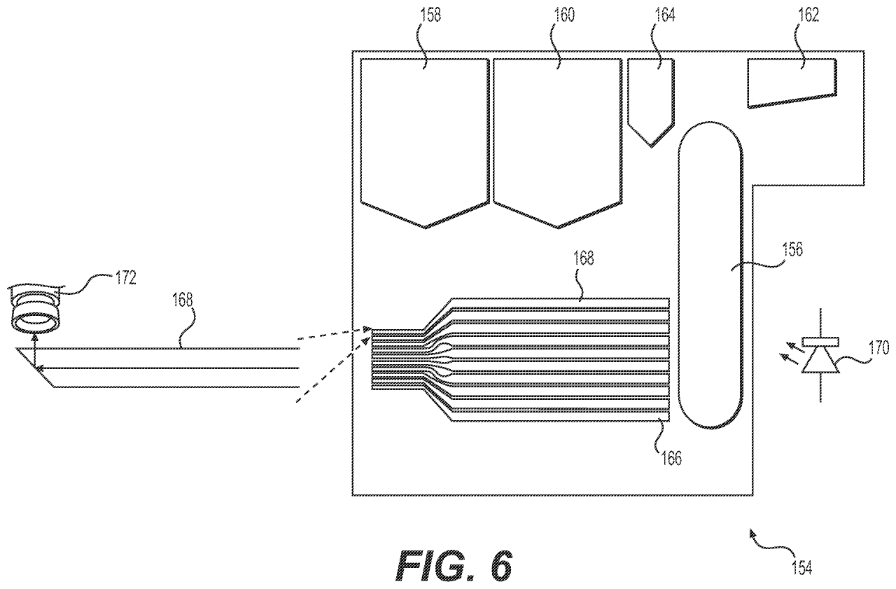

Cartridges can be designed and configured to support other types of target measurement systems. Similar to the cartridge shown in FIG. 1B, the cartridge shown in FIG. 6 is configured to support optical measurement. As with the IFC cartridge described above, optical measurement cartridge 154 can couple to the fluidics and pressure handling system of the larger system via ports on the cartridge. Valves, fluidic lines, and pressure lines are included in the cartridge to assist with the flow of fluid therethrough. The positioning of all optical cartridge components can vary. Like the cartridge shown in FIG. 4, optical measurement cartridge 154 can include a filtering chamber 156, a target sample fluid input well 158, a waste well 160, a backflush chamber 162, a mixing well 164, at least one test compartment 166, and at least one control compartment 168. Sample filtration and movement to the compartments 166, 168 can proceed in a similar fashion as was described for FIG. 4. The compartments 166, 168 are configured to act as light guides, transmitting light from light source 170, through the channels containing target sample fluid, and to a detector 172. Optical transmittance, or optical density, can be calculated as described above and associated with growth of a target species.

Cartridges can also be designed for other types of measurement devices that could be included with the target sensitivity testing system. For example, changes in nutrient or metabolite concentration could be measured in place of or in addition to optical or impedance properties. In another example, changes in electrical charge of the target sample fluid (for example, if the fluid contained charged nanoparticles) can be measured instead of or in addition to any of the other types of measurement described herein.

### Methods for Determining Target Sensitivity to a Therapeutic Formula

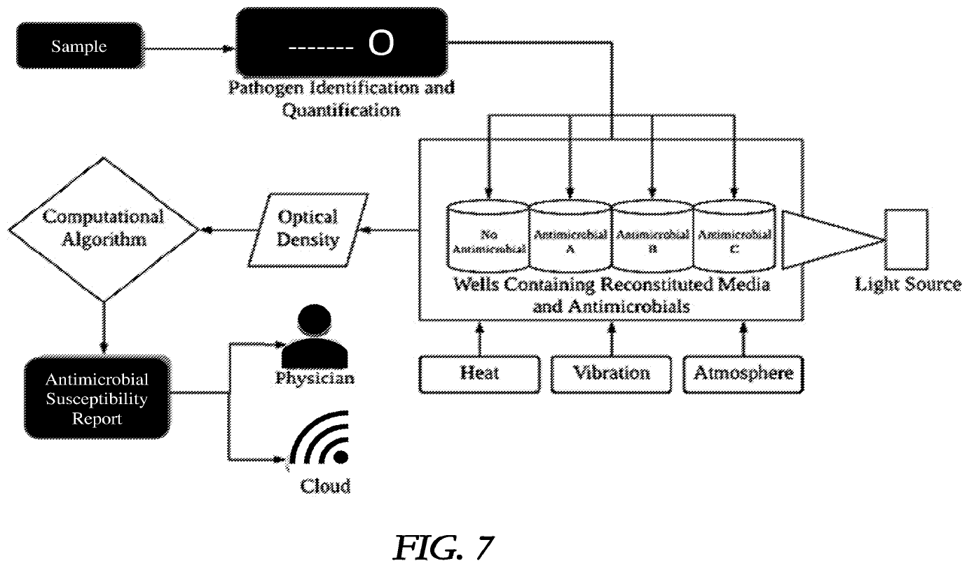

An example flow chart of the methods disclosed herein is presented as FIG. 7. The flow chart is presented for illustrative purposes only, and is not meant to limit the invention. Certain steps may be added or removed from flow chart presented below, or the steps can be performed in an alternate order, without detracting from the inventive concepts disclosed herein. Likewise, certain steps described in the description below can be added to, removed, or rearranged without detracting from the inventive concepts disclosed herein. The flow chart shows the optical measurement steps, but the optical measurement steps and system components can be replaced with other types of measurement steps and system components, including, but not limited to, those associated with IFC, those associated with measuring the concentration of a nutrient or metabolite in the target sample fluid, and/or those associated with measuring changes in electrical charge of the target sample fluid.

In an aspect, a method of the present disclosure can be operated within the same or within a similar device that is used to identify the target. Such a combined device is shown in FIG. 8, where the identification module 49 and the sensitivity module 51 are identified. The sensitivity module 51 can be constructed according to the principles outlined in the systems section, above. In an aspect, the elements of a kit using the stem-loop captor method described in International Publication No. WO 2017/218858 can be modified to include a dispensing apparatus and one or more incubation compartments and configured such that a light source can be activated to transmit through a compartment and be detected for the target sensitivity assay.

The target sample can optionally be prepared by concentrating, filtering, or any other sample preparation steps. In some embodiments, this may be performed manually using known laboratory techniques. Or, advantageously, sample preparation can be handled automatically by a sample preparation module that is in fluid communication with the identification and/or sensitivity modules described below. The sample can be automatically prepared separately for each of the identification and sensitivity modules, or the sample can be prepared as a whole and delivered in series to the identification module and then to the sensitivity module (after target identification and quantification). If target identification and quantification is being performed using an alternative to the CAPTURE stem-loop captor method, then the sample may be prepared manually or automatically and delivered directly to the sensitivity testing module.

In some embodiments, the target species can be identified prior to testing the sensitivity of the target species to therapeutic formulas. The concentration of the target species in the biological sample can also be determined to assist with calculations. Concentration can be determined during the target identification steps using pump and vessel volumes. It can be advantageous to carry out these steps using the CAPTURE stem-loop captor method described above and in WO 2017/218858. However, target identification and quantification may be carried out by any known method without affecting the novelty of the inventive concepts described herein for assaying target sensitivity to therapeutic formulas.

In an aspect, a sample may be divided such that a portion of the sample is assayed using an identification module 49 and another portion of the sample is assayed for sensitivity to one or more therapeutic formulas using the sensitivity module 51. Alternatively, a separate sample can be collected and assayed separately for identification and sensitivity. In an aspect, prior identification of a target in a sample using the stem-loop captor method can advantageously allow for the use of incubation conditions that favor the growth of the identified target species. In an aspect, prior identification of a target in a sample using the stem-loop captor method can allow for the incubation of the sample with one or more therapeutic formulas that can be appropriate therapeutic agents for the identified organism or target. In an aspect, the sensitivity of a target to a therapeutic formula can be assayed prior to, concurrently with, or after the identification of the target.

In another step of the method, the target sample fluid is introduced to a disposable cartridge 11, such as the one shown in FIG. 1B. The cartridge can come preloaded with therapeutic formula, with or without components such as growth medium to foster multiplication of the target. Alternatively, the end user can add the therapeutic formula and/or growth medium at the point of the assay. In some embodiments, the flow of target sample fluid can be automatically directed from the identification module 49 to the sensitivity module 51, as discussed above. In the automated embodiment, the sample fluid is inserted into the cartridge 11, directly or indirectly (i.e. via insertion port 33 shown in FIG. 1B), and the sensitivity module 2 will direct the sample to appropriate compartments. Automated fluid handing advantageously reduces the time spent handling the sample and eliminates potential sources of errors. Automated fluid handling methods can include using fluidic channels, pumps, vacuum, gravity feeds, valves or any other method to direct flow to certain compartments.

In another step of the method, at least a portion of the target sample fluid is placed into at least one test compartment comprising a first therapeutic formula and components that foster the multiplication of the target (such as, for example, a target growth medium). To assay the sensitivity of the target to various potential therapeutic formulas, or varying concentrations of a potential therapeutic formula, the target sample fluid can be placed into two or more test compartments that include one or more therapeutic formulas and components that foster the multiplication of the target. In some embodiments, the target sample fluid is shared to a control compartment that does not include a therapeutic formula but does include components that foster the multiplication of the target. The target sample fluid can be handled and directed throughout the cartridge of the sensitivity module 51 in an automated fashion. In some embodiments, certain compartments in the cartridge will be mechanically activated to test for specific formulas, based on firmware directions resulting from the identification of the CAPTURE assay. The growth medium can be dispensed from a single vesicle into the compartments prescribed.

In another step of the methods, environmental factors (such as heat, vibration, and gas levels) can be controlled to optimize the conditions for target multiplication. In an aspect, the incubation of the target sample with or without one or more therapeutic formulas can be carried out at a range of temperatures, including but not limited to temperatures ranging from 15 C to 50 C, such as 37 C or 42 C. In an aspect, the incubation of the target sample with or without one or more therapeutic formulas can be carried out at a range of atmospheric conditions, including but not limited to percentages of oxygen below normal atmospheric oxygen levels and percentages of carbon dioxide above normal atmospheric carbon dioxide levels. In an aspect, such percentages of oxygen can be, but are not limited to, from 0 percent to 20 percent, and such percentages of carbon dioxide can be, but are not limited to, from 0.04 percent to 10 percent. In an aspect, the incubation of the target sample with or without one or more therapeutic formulas can also include incubation of the target sample with one or more additional compounds that would normally promote the growth of a cell or organism, such as amino acids, sugars, yeast extracts, or other such nutrient compounds. In an aspect, the incubation of the target sample with or without one or more therapeutic formulas can also include incubation of the target sample with a buffer that maintains the characteristics of the target sample, such as in pH or small molecule composition.

In some embodiments of the methods, optical properties of the target sample fluid can be measured. The optical properties, such as optical density or transmittance, can be measured continuously or periodically over a time period using a light source, and detector, and a light path length that is longer than the conventional, industry standard light path length. The conventional approach in optical property measurement is to use small wells or cuvettes. The approach disclosed herein is designed for a longer path length, and customized into a cartridge with a small footprint. The long light path length exponentially improves time to detection. The long light path maximizes path length while minimizing volume. The longer light path can be accomplished with vessels, and/or including use of mirrors and/or reflective compartment siding, as discussed above.

In another step of the methods, the target sample fluid can be moved throughout the cartridge using pumps that couple with the pressurization system. In some embodiments, the pumps and the pressurization system are pneumatically operated, but other forms of pressure can also apply. The target sample fluid is moved through the measurement system such that measurements can be performed over time. Changes in the measurement properties are associated with target species growth and the processor is used to construct growth curves over time for the various test and control compartments of the cartridge.

Referring back to FIGS. 3-5, a method of IFC measurement will be described. The chambers can incorporate pumps to slowly move the sample and to keep the sample mixed. The measurement from an IFC property will include taking multiple measurements of impedance over a time period to produce a growth curve that can be compared to the threshold growth curve from a control chamber without a therapeutic formula. Referring now to FIG. 4, the target sample fluid is passed from the sample fluid input well 118, to the target sample fluid inlet 120, through the filtering chamber 116, out the waste outlet 122, and to the waste well 124. The target species is caught in the filtering chamber, with the remainder of the target sample fluid moving into the waste well 124. A backflush inlet 126 can accept a solution, such as target growth media, from a backflush chamber 125, to backflush the target species back out the filtering chamber 116 and out of a backflush outlet 128 of the filtering chamber 116. In some embodiments, filtered target sample fluid then travels from backflush outlet 128 to mixing well 130, which can receive additional target growth media and/or electrolytic solution. Calculations can be performed to add the appropriate volume of fluid to adjust the species concentration to an optimal range for the IFC assay. The target sample fluid can then be moved to compartments 132, 134 and through channel 140 repeatedly (past IFC electrodes), with IFC measurements being taken over a time period. Changes in impedance of the target sample fluid over time can be associated with multiplication of the target species. The sensitivity of the target to the one or more therapeutic formulas can generally be determined within a time frame of from about 5 minutes to about 360 minutes.

In another aspect, the method includes measuring an optical property of the target sample fluid in the at least one test compartment under conditions favorable for target multiplication or growth. For example, during the incubation of a sample, an optical transmittance measurement can be taken on the sample by shining a light source through the sample and measuring the amount of light captured by a detector. The optical transmittance measurement can be repeated at time intervals during the incubation of a sample. The method can further include measuring a control optical property of the target sample fluid in the control compartment under conditions favorable for target multiplication or growth and in the absence of the at least one therapeutic formula, and using the control optical property in a calculation to determine the threshold optical property. The optical property can be measured at multiple wavelengths. The wavelength or wavelengths of the light used to measure the optical property can be between from 200 nanometers to 10,000 nanometers, in some embodiments (from UV, to visible light, to IR). The sensitivity of the target to the one or more therapeutic formulas can be determined within a time frame of from about 5 minutes to about 360 minutes.

In some embodiments, the concentration of a nutrient (such as, but not limited to, glucose) in the target sample fluid can be measured continuously or periodically to provide an indicator of target species multiplication (consumption of the nutrient indicates multiplication of the target). In some embodiments, the concentration of a metabolite in the target sample fluid can be measured continuously or periodically to provide an indicator of target species multiplication. Metabolite herein refers to any substance produced during metabolism of a target species. The metabolite that is measured can be, for example, a short-chain fatty acid, an organic acid, a vitamin, a bile salt, a polyphenol, a lipid, or an amino acid. In some embodiments, an increased level of a metabolite indicates multiplication of the target. In some embodiments, changes in the electrical charge of the target sample fluid can be measured over time to indicate multiplication of the target (for example, if the target sample fluid were loaded with charged nanoparticles.

The system can include more than one type of measurement device, and the methods can include taking measurements in one or more different ways. In some embodiments, use of two or more measurement devices can minimize error and can provide backup in the unlikely event of the failure of a first measurement device.

In another step of the methods the multiple measurements taken over the given time period can be used to construct measurement curves for each compartment. These measurement curves translate to target growth curves for test compartments and control growth curves for control compartments. For example, a decrease over time in the optical transmittance of a sample incubated with or without one or more therapeutic formulas indicates that the target in the sample is multiplying or growing in the sample. Little or no change over time in the optical transmittance of a sample incubated with or without one or more therapeutic formulas indicates that the target in the sample is not multiplying or growing in the sample. An analysis of the change over time in the optical transmittance of a sample with or without one or more therapeutic formulas indicates whether the target in that sample was multiplying or growing.

Each measurement curve from a test compartment is compared to one or more measurement curves from a control compartment. The control compartment readings are used to set threshold values to which the test compartment readings are compared. The software calculates the deviations between test and control readings, and significant deviations are used to indicate potentially successful therapeutic formulas (i.e., formulas that prevented target growth).

In another step of the methods, an indicator, such as a reading on a local user interface, can report which compartments had effective therapeutic formulas versus ineffective therapeutic formulas. Sensitivity and resistance results may also be reported, wired or wirelessly, to a tablet, remote display, electronic medical records, cloud-based databases or other archiving options.

Assay results comprising data on the sensitivity and/or resistance of the target to various therapeutic formulas can also be stored to the memory of an adaptive machine learning tool, and the assay results are used by the adaptive machine learning tool in longitudinal and/or geographic analyses of target sensitivities and resistances to particular therapeutic formulas. For example, the adaptive machine learning tool may access a cloud-based database of target sensitivity assays pooled by multiple points of care. The assay results can be accessed by the adaptive machine learning tool at a later point in time to predict changes in the sensitivities and resistances of particular targets to various therapeutic formulas. In this way, the evolution of target resistances can be monitored from a public health perspective. Furthermore, the adaptive machine learning tool may use pooled target sensitivity/resistance data to predict epidemics, pandemics, or early stage outbreaks. The adaptive learning tool can also be used to show the likelihood of antibiotic misuse, families of antibiotics with similar responses, and the financial cost of various antibiotic options.

### EXAMPLES

The target sensitivity determination system disclosed herein can be used to test for multiple syndromes and organisms. The examples will describe the target sensitivity determination system in the context of urinary tract infections (UTI). This technology can significantly reduce patient suffering and the risk of complications due to UTI; lost work time; and the guess work for antibiotic prescription that is still the current standard of care. Urine for UTI is the most commonly handled sample and UTIs are the second most common type of infection accounting for more than 10 million medical visits annually and are expected to rise as the population increases and ages. By age 32, half of women will have contracted a community-acquired UTI. Increasing incidence with age makes it the most common infection in elderly women. UTIs are also the second leading cause of pediatric medical visits but are often difficult to diagnose because of the unusual symptoms, such as irritability or lack of appetite. In the elderly, 20-30% of infections in long-term care (LTC) residents are due to UTIs. Although prolonged use of catheters has decreased markedly in the last decade, catheter-associated UTIs are still the most common type of healthcare acquired infection (HAI) and cause half of bacteremia (blood infection) occurrences in LTC facilities.

Many uropathogens are resistant to commonly used antibiotics, but most patients are still treated empirically with a drug that may or may not be effective while they wait for culture results. Rapid diagnosis of UTI is essential for proper treatment, antimicrobial stewardship, and prevention of life-threatening complications. Culture results generally take 2-3 days, during which time an unnecessary or wrong antibiotic may have been prescribed. Cheap, rapid dipstick strips to identify the presence of blood, nitrites, and leukocytes in urine are routinely used by medical staff and are now available for purchase as at-home screens. However, false positives and negatives are common, and elevated levels in the elderly confound their utility Amplification-based molecular diagnostics have largely ignored the UTI market due to the “false positive” problem of amplifying contaminating urogenital flora found in most urine specimens. Misdiagnosed and untreated UTIs result in extended patient suffering and can lead to kidney damage, sepsis, and death.

Some AST methods recently approved or in development follow the growth of smaller populations of bacteria and generalize from their behavior the AMR status of the pathogen (Baltekin et al., Point-of-care antibiotic susceptibility test, PNAS 2017; 114:9170). Although urine is by nature polymicrobial, the overabundance of uropathogens compared to commensal flora in positive UTI samples allows initial changes in bacterial abundance to reflect the growth of the uropathogen itself for separating negative and positive urines (Puttaswamy et al., A Comprehensive Review of the Present and Future Antibiotic Susceptibility Testing (AST) Systems, Arch Clin Microbiol. 2019; 9: 83). Comparison of these initial changes in growth between samples with and without antibiotics would allow AMR to be determined rapidly; however, products in this space do not determine the identity or concentration of the uropathogen and therefore cannot be used for AST. Additionally, most AST methods are still being designed for laboratory settings. A large unmet market remains for rapid, affordable UTI pathogen ID and AST at near-patient locations.

The solutions disclosed herein (target identification+target sensitivity determination system) will identify over 95% of community-acquired uropathogens, including the components of mixed infections, and distinguish true infection from commensal flora. The device will first identify 10 common uropathogens directly from urine (single or mixed infections). If the ID test finds pathogens≥10⁴ CFU/mL (commonly accepted cut-off for ambulatory UTI), then the companion AST will be run against 7 common oral antibiotics appropriate to treat UTI. Testing at clinics, physicians' offices, nursing homes and other POC sites will significantly shorten the time to result for correct patient treatment and allow a revenue stream to those POC sites. The time for identification+target sensitivity testing (about an hour) still allows prescriptions to be promptly called in for patients with positive results. With the described microarray-based identification technology, the assay is able to identify hundreds of pathogens simultaneously.

It is noted that certain methods described below in reference to Example 1 may be used with the technology of Example 2, and that certain methods described below in reference to Example 2 may be used with the technology of Example 1.

### Example 1—Testing Target Sensitivity Using Spectrophotometry in the Context of UTI

Methods for Example 1:

To prepare a spiked bacterial sample, 1 mL of an overnight culture was diluted into 25 mL of warm cation-adjusted Mueller Hinton broth, allowed to grow to log phase at 37 C in a shaking incubator, and counted using a hemocytometer. The bacteria were then diluted in additional warm broth to a final concentration of 1E5 to 8E5 CFU/mL. A 2-mL aliquot of the bacterial solution was added to a disposable 1-cm cuvette containing a pre-dispensed volume of water, diluent and antibiotic according to the experiment at hand. The fluids were mixed by pipetting. A no-antibiotic control was used in each experiment, which contained only the appropriate volume of water and diluent. A set of three cuvettes, such as in 41 or 43a, 43b or 43c, containing identical mixtures were assembled side-to-side into a three-centimeter light path and inserted into the prototype sensitivity module shown in FIGS. 2A and 2B. A total of four sets of cuvettes could be examined in the prototype at one time. The prototype maintains the cultures at 37 C with gentle agitation to mitigate the effects of bacterial settling. Optical transmissions through each set of cuvettes were taken every 5 seconds for the no-antibiotic control and the experimental cuvettes. The bacteria were allowed to grow from 10 minutes to two hours depending on the antibiotics and/or bacteria being tested. The final curves were analyzed to determine whether the bacteria were sensitive or resistant to each drug or drug combination.

For methods including identification by the CAPTURE assay, the pathogen concentration will be quantified during CAPTURE and will inform the volume of the original urine sample needed for sensitivity testing. Once loaded into the sensitivity module, this volume will be passed across a filter and then backwashed with cation-adjusted Mueller Hinton broth (stored in the cartridge in powdered form and reconstituted prior to testing) to give 2E5 to 8E5 CFU/mL. After mixing, a small volume (≤500 uL) will be automatically dispensed into the appropriate growth chambers containing lyophilized antibiotics and a no-antibiotic control.

A list of antibiotics tested using the methods disclosed herein is given in Table 1. DMSO=dimethyl sulfoxide; PBS=1× Phosphate Buffered Saline, pH 7.2. Dried antibiotics may be provided/preloaded within the compartments of the cartridge.

TABLE 1

| | | | Final |
|---|---|---|---|
| | | Initial | Concentration |
| Antibiotic Used | Abbreviation | Diluent | (μg/mL) |
| Ampicillin | AMP | H₂O | 8 |
| Amoxicillin/Clavulanate | AMC | H₂O | 8/4 |
| Trimethoprim/Sulfamethoxazole | STX | DMSO | 2/38 |
| Ciprofloxacin | CIP | H₂O | 4 |
| Fosfomycin | FOS | H₂O | 64 |
| Levofloxacin | LEV | H₂O | 2 |
| Nitrofurantoin | NIT | DMSO | 32 |
| Tetracycline | TET | PBS | 16 |

In the proof-of-concept device used to generate the data provided in this example, a 3-cm light path and lasers at 405 nm and 532 nm were used. Antimicrobial resistance profiles of 17 clinical isolates of 11 uropathogens were followed from 20 minutes to three hours, against 8 antibiotics, both lytic and static.

Results for Example 1:

Table 2 shows that the methods described herein have been successfully used to both identify and determine the AST profile of a number of clinically important bacterial pathogens, all of which can cause UTI.

TABLE 2
Preclinical Validations Confirm Approach

| | Strains ID | Strains AST |
|---|---|---|
| Organism | Confirm | Confirm |
| E. coli | 21 | 3 |
| K. pneumoniae | 5 | 2 |
| P. mirabilis | 3 | 2 |
| E. faecalis | 1 | 1 |
| S. aureus | 1 | 1 |
| P. aeruginosa | 4 | 2 |
| S. saprophyticus | 1 | 1 |
| E. cloacae | 1 | 1 |
| C. freundii | 2 | 1 |
| M. morganii | 1 | — |
| Group B Strep | 1 | — |
| TOTAL | 41 | 14 |

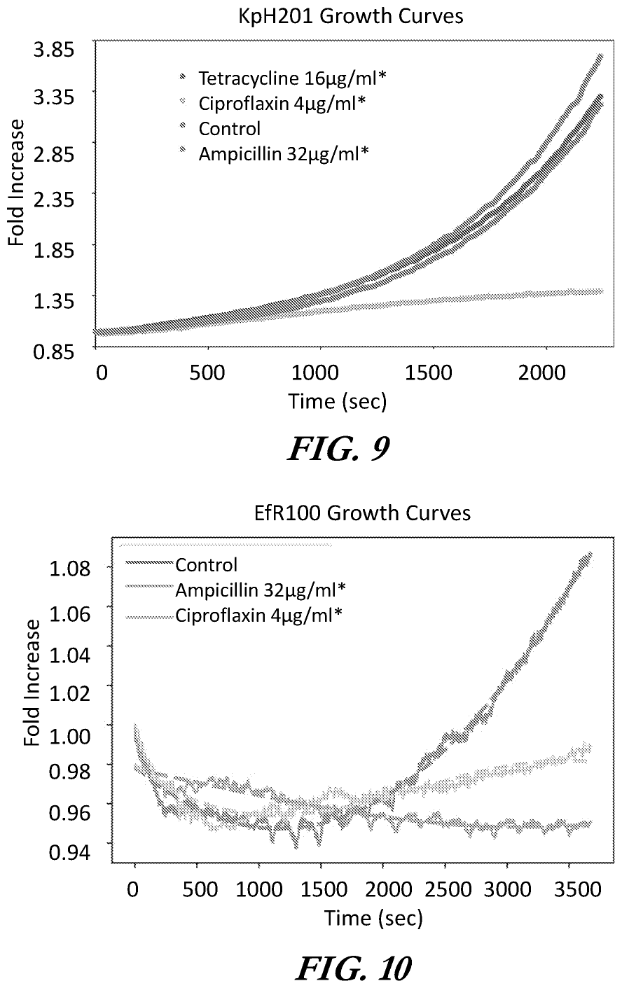

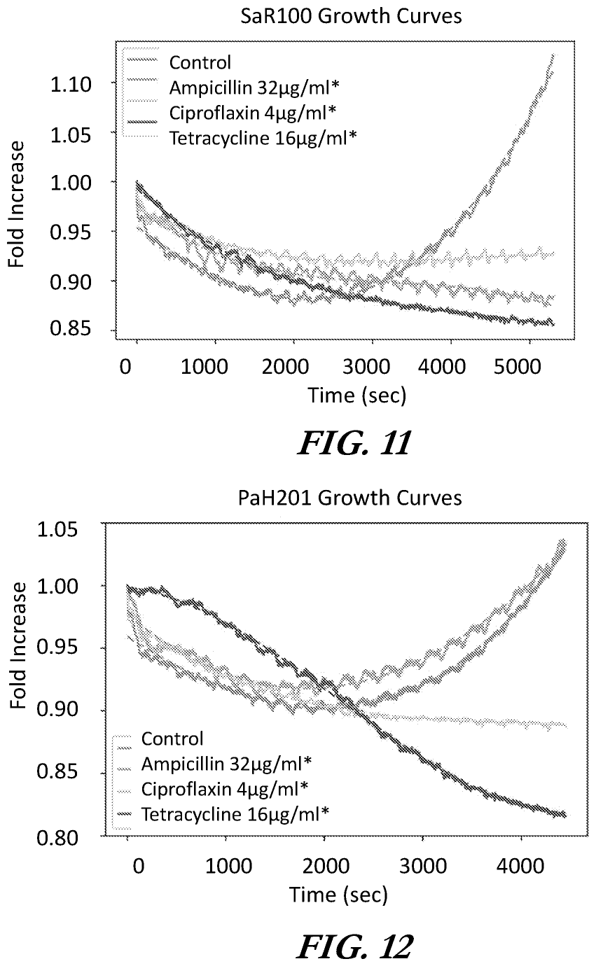

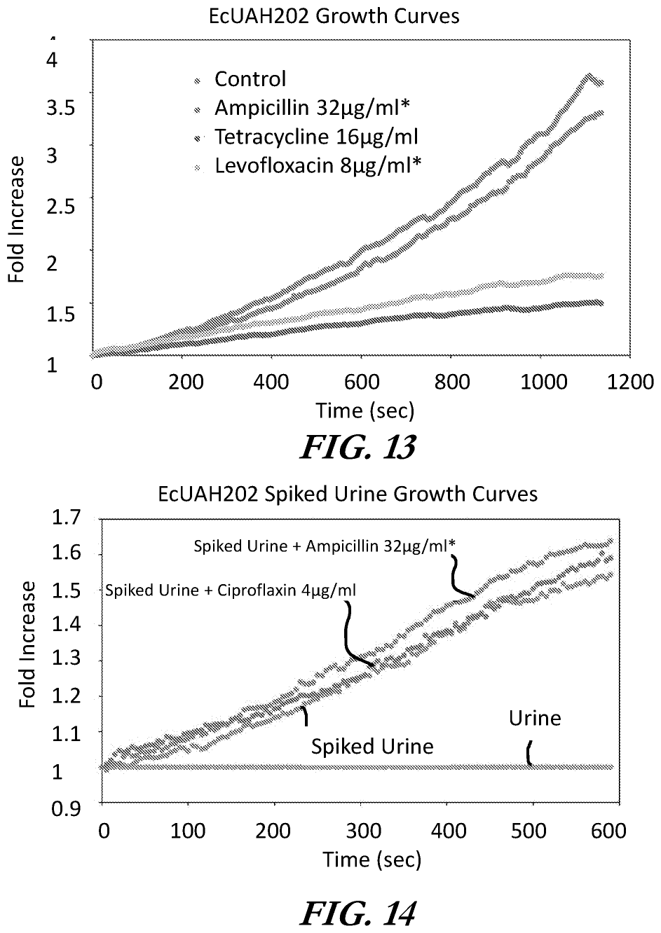

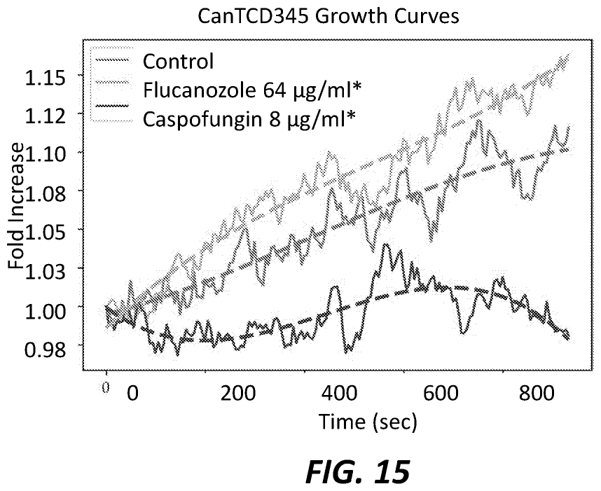

The growth profiles of 6 reference isolates of the 7 bacterial organisms on the CAPTURE UTI panel are given in FIGS. 9-15. These growth profiles were measured using the methods disclosed herein and show growth of the same organism when exposed to clinically relevant breakpoint concentrations of antibiotics. All curves denoted with a * indicate confirmation of bacterial culture determined antibiotic susceptibility or resistance. Confirmation is from a conventional microbiology lab as part of their standard of care results for UTI antimicrobial sensitivity testing for these samples. Organismal growth or lack of growth was determined by comparison of instantaneous rates of change for each growth curve over time. FIGS. 10-12 and 15 depict growth curve data and a fitted 3^{rd} order polynomial in order to mitigate noise interference with growth calls. This also serves as a method of algorithmically plotting instantaneous rates of change, enabling earlier semi-automated growth/no-growth antibiotic susceptibility calls. In all figures below, the y-axis values are fold increase in optical density in relation to each well's initial time point according to the equations shown below.

$$\begin{matrix}S ( t ) = OD - background & Equation 1 \\ v ( t ) = \frac{S ( t = 0 )}{S ( t )} & Equation 2\end{matrix}$$

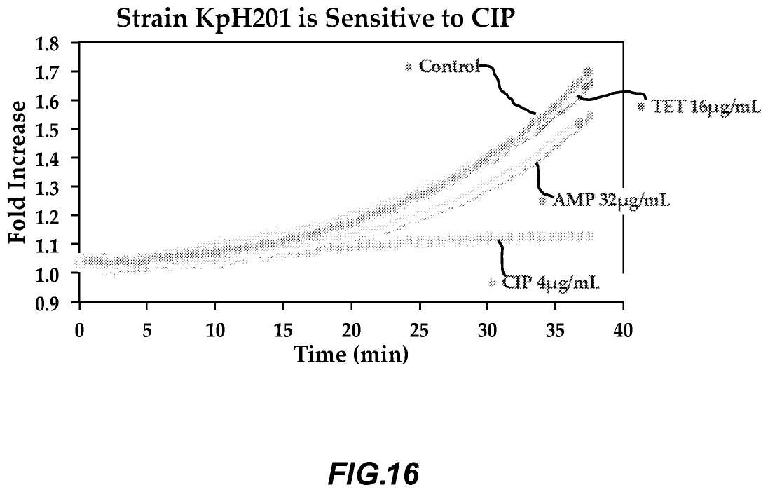

FIG. 16 shows results of an AST assay on KpH201, taken at 5 second intervals for 37 minutes, using the methods disclosed herein and the system shown in FIGS. 2A and 2B. The data show that the KpH201 strain is sensitive to CIP.

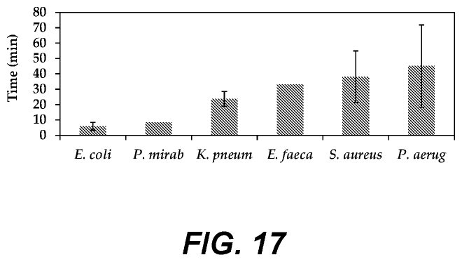

As shown in FIG. 17, the average time to result was significantly less than 1 hour for most runs. Initially, 70 runs comparing 181 drug exposures to no-drug controls were evaluated and the performance of the device modified. Fifty-one subsequent assays were evaluated against culture results, primarily with nitrofurantoin (NIT), ampicillin (AMP), amoxicillin (AMC), cefazolin (CFZ), trimethoprim/sulfamethoxazole (SXT), tetracycline (TET), and levofloxacin (LEV) with most being tested in duplicate or triplicate. As shown in Table 3, no false resistances (major errors) or false sensitivities (very major errors) were encountered with these drugs.

TABLE 3
No Errors in AST Results

| | # of | Major | Very Maj. |
|---|---|---|---|
| Drug | Strains | Error | Error |
| NIT | 11 | 0 | 0 |
| AMP | 10 | 0 | 0 |
| AMC | 4 | 0 | 0 |
| CFZ | 2 | 0 | 0 |
| SXT | 5 | 0 | 0 |
| TET | 13 | 0 | 0 |
| LEV | 6 | 0 | 0 |
| TOTAL | 51 | 0 | 0 |

### Example 2—Testing Target Sensitivity Using Impedance Flow Cytometry in the Context of UTI

The Clinical & Laboratory Standards Institute (CLSI) provides guidelines for antibiotic susceptibility testing, which requires laboratories to know the identity of the pathogen and to standardize the inoculum for antibiotic testing (10⁵≤10⁶ CFU/mL) (Clinical and Laboratory Standards Institute. Performance Standards . . . 29^{th} edition CLSI Supplement M100; 2019). These requirements and the mixed populations found in direct specimens create a challenge for developing rapid near-patient AST methods. The UTI ID assay disclosed herein is capable to report both the identity and concentration of uropathogen(s) in orders of magnitude from 10⁴ to ≥10⁶. Using these data, an appropriate volume of the original specimen can be placed into a fully automated AST cartridge where it will first be passed through a system of filtration membranes to purify bacteria away from host cells. The rinsed bacterial component will then be backflushed into cation-adjusted Mueller Hinton Broth (CAMHB), mixed in a holding chamber to give the required CFU/mL, and distributed into chambers containing dried antibiotics at appropriate breakpoint concentrations, and control chambers with no drugs. Initial quantitation in each chamber will control for bacterial concentrations followed by incubation and the monitoring of cell counts for up to 2 hours. An algorithm applied to the resulting growth curves in real-time allows AST results to be reported as Sensitive (S) or Resistant (R). Samples for which the results are inconclusive will be reported as such.

The Coulter principle for counting and typing blood cells and bacteria was developed in the 1950's (Coulter, W. H. High speed automatic blood cell counter and cell size analyzer. Proc Natl Electron Conf. 1956; 12) (Kubitschek H. Electronic Counting and Sizing of Bacteria. Nature. 1958; 182:234) and was proposed for monitoring antibiotic effects as early as the 1980's (Seydel et al., A simple, fast and inexpensive kinetic method to differentiate between total and viable bacteria using Coulter counter technique. Arzneimittelforschung. 1980; 30:298) (Schulz et al., Relationship between the bactericidal and bacteriolytic activity of cephalosporins and changes in the cell volumes of Escherichia coli cultures. Infection. 1985; 13:235). A Coulter counter passes particles in a weak electrolyte solution through a small aperture between two chambers containing electrodes, and the impedance between the electrodes changes when a particle passes through the aperture. Flow cytometry variations of the Coulter method have electrodes placed around or adjacent to a small channel connecting the two compartments (see FIG. 3A, for example.) Computer analysis of impedance changes as particles pass through the tube allows the discrimination of particles of various shapes and sizes; gram-negative rods can be distinguished from cocci, for example (Swanton et al., Experiences with the Coulter counter in bacteriology. Appl Microbiol. 1962; 10:480). Modern manufacturing technologies have facilitated the production of small, disposable impedance flow cytometry (IFC) devices (Sun T. et al., Single-cell microfluidic impedance cytometry: a review. Microfluidics Nanofluidics. 2010; 8:423) (Cheung, K C et al., Microfluidic impedance-based flow cytometry. Cytometry. 2010. Part A. 77:648) (Chen, J., et al. Microfluidic Impedance Flow Cytometry Enabling High-Throughput Single-Cell Electrical Property Characterization. Int. J. Mol. Sci. 2015; 16: 9804). A low electrolyte concentration is critical to the impedance measurements, and CAMHB should prove an effective solvent for this technique. IFC can therefore be used to monitor the concentration and growth of the identified uropathogens in each chamber of the proposed AST cartridge. The innovative approach of combining direct from urine AMR testing with growth monitoring that tracks bacterial morphology will facilitate an affordable and rapid near-patient solution to guide UTI treatment.

Feasibility of Determining Uropathogen AMR Direct from Urine Using Impedance Flow Cytometry (IFC)

A pair of IFC devices, for example BactoBox (SBT Instruments, Denmark), enables the monitoring of antibiotic effects during growth. The two most common Gram-negative uropathogens, Escherichia coli and Klebsiella pneumoniae, and two common Gram-positive uropathogens with differing growth characteristics, Enterococcus faecalis (individual cocci and chains) and Staphylococcus aureus (individual cocci and clusters) are tested. These initial tests use 1-mL samples of stock strains spiked into CAMHB at concentrations of 105≤106 CFU/mL. As a control for bacterial concentration, serial dilution and plating is performed for each test. Samples are tested in pairs: one spiked with antibiotics and the other with diluent only. Both bacteriolytic (NIT, AMP, AMC, CFZ, LVX) and bacteriostatic (SXT, TET) antibiotics are included. The samples are incubated at 35° C. for up to 2 hours with mixing. At desired time intervals, each entire 1-mL sample is passed through an IFC device in less than 2 minutes; a separate IFC device is used for each sample. The cell count and total cell volume as a function of time is plotted for each pair of samples and growth patterns analyzed for sensitivity or resistance. The results are matched to equivalent samples monitored spectrophotometrically and also compared to the known AMR of these strains.

The filtration and backflush of spiked urine can be characterized. Commercially available pathogen-negative urine is spiked with the four uropathogens at levels seen in UTI (from 1×10⁴ to 1×10⁸.) Appropriate volumes of these samples is passed through a 5-μm pore nylon prefilter to separate out host cells, then through a 0.2-μm pore nylon filter to capture bacteria. The 0.2-μm filter is rinsed with CAMHB to remove traces of urine, and backflushed with additional CAMHB. The volume of recovered backflush and the percent recovery of each pathogen from the pathogen-negative urine over the tested concentration range is determined in triplicate. These results inform the volume of sample at a variety of UTI conditions needed for AST testing. If the volume of a low positive sample (1×10⁴ CFU/mL) needed to achieve at least 1×10⁵ CFU/mL for AST is impractically high, then very low pathogen UTI samples may only obtain ID.

The ability to filter and backflush pathogen-positive samples is verified similarly to that of pathogen-negative samples (spiked negative urine may not be as complex as positive urine samples because urine samples from UTI patients often contain leukocytes, red blood cells and proteinaceous casts). Therefore, discarded, deidentified urine samples from ambulatory, non-catheterized patients are procured under IRB from local clinics. The backflushed samples from the pathogen-positive urine are used for impedance flow cytometry monitoring of antibiotic growth responses, as described above, with the results compared to traditional AST methods. The ability to distinguish Gram-negative rods from Gram-positive cocci or rods (Lactobacilli) is determined, as is the performance of the method in monitoring changes in cell volume and cell number.

Design and Manufacture of a Cartridge and Instrument for the Automated Testing of Positive UTI Samples.

The AST cartridge leverages the form factors of the ID cartridge and its filtration platform (FIG. 8, for example). Molding and fabricating the features for the 10 AST growth chambers is affordable for an inexpensive cartridge when produced in volume. The AST cartridge will contain all necessary fluids with the antibiotics freeze-dried in the assigned chambers (Table 4) for sample-to-answer automated testing. Only levofloxacin needs to be in two chambers at two different concentrations for the uropathogens on this panel.

TABLE 4

| AST Cartridge Chambers: | | | | | | | | |
|---|---|---|---|---|---|---|---|---|
| No antibiotic in Chambers 1, 10 | 2 | 3 | 4 | 5 | 6 | 7 | 8 | 9 |
| Pre-loaded Antibiotics | NIT | AMP | AMC | CFZ | SXT | TET | LVX | LVX |
| Sensitivity Breakpoint (μg/mL) [16] | 32 | 8 | 8/4 | 16 | 2/38 | 4 | 1 | 2 |

Pathogens ID'ed by companion assay and antibiotics tested for each

| Escherichia coli | ✓ | ✓ | ✓ | ✓ | ✓ | ✓ | | ✓ |
|---|---|---|---|---|---|---|---|---|
| Klebsiella species (with E. | ✓ | | ✓ | ✓ | ✓ | ✓ | | ✓ |
| aerogenes) | | | | | | | | |
| Enterobacter species | ✓ | ✓ | ✓ | ✓ | ✓ | ✓ | | ✓ |
| Proteus species | ✓ | ✓ | ✓ | ✓ | ✓ | ✓ | | ✓ |
| Pseudomonas aeruginosa | | | | | | | | ✓ |
| Staphylococcus aureus | | | | ✓ | ✓ | ✓ | ✓ | |
| Staphylococcus saprophyticus | CLSI: AST not advised on urine. | | | | | | | |
| Staphylococcus lugdunensis | | | | ✓ | ✓ | ✓ | ✓ | |
| Streptococcus agalactiae (Group B strep) | CLSI: AST not required. | | | | | | | |
| Enterococcus species ✓ | ✓ | ✓ | | ✓ | | ✓ | | ✓ |

A preliminary design of a laminate-build cartridge is shown in more detail in FIG. 4 (for simplicity, fluidic channels and pneumatic channels are not shown). The specimen is loaded into the Input Well and passed through the Filter Stack, with the bulk urine passing into the Waste Well. The Filter Stack is rinsed and backflushed with CAMHB and the flushed bacteria enters the Mixing Well. From there, 100 microliters are pumped into each of the ten bi-lobed Incubation Chambers. The fluid is periodically pumped between Upper and Lower lobes, with the bacteria in each chamber counted in turn as it passes through the connecting channel. This method for performing IFC involves a Long Channel that allows the passing organisms to achieve a uniform flow. Measuring the impedance in each of the 10 Incubation Chambers in turn minimizes the electronics needed in the instrument. Alternating the positive and negative electrodes across the channel in the anticipated electronic configuration improves signal detection (FIG. 5) (Caselli, F., et al., A novel wiring scheme for standard chips enabling high-accuracy impedance cytometry. Sensors and Actuators B: Chemical. 2018: 256:580). In FIG. 5, partial pneumatic lines extend beneath the lobes (not shown). The upper and lower lobes each act as a pump to slowly move the fluid through the channel to the other lobe. This movement allows each sample to be continually mixed as the IFC measurements are performed. Disturbances in the electric field are analyzed to quantify the number and size of bacteria throughout the incubation period by passing the fluid through the channel through upper and lower lobes using pneumatic pressurization.

Methods for Providing Antibiotics.

Antibiotic solutions can be manually prepared and loaded into devices and/or cartridges. Alternatively, chemical engineering techniques and equipment can be used to stabilize, dry, and reconstitute desired antibiotics within the cartridge so that the cartridge can be provided to the user with antibiotics included.

The AST Cartridges and Instrument are Tested Against Spiked Urine Samples.

The cartridges and instrument are tested in a laboratory setting. For proof-of-principle testing, urines spiked at various concentrations with one of the 4 representative organisms listed above (E. coli, K. pneumoniae, S. aureus, and E. faecalis) are analyzed by research technologists. Discarded, deidentified negative urine samples from ambulatory, non-catheterized patients are collected under IRB and spiked with three concentrations of each organism: the lowest expected concentration possible for AST, a middle range of 1×10⁶ CFU/mL, and a high range of 5×10⁷ to 1×10⁸ CFU/mL. Replicates of the same organism spiked into the same urine sample are run in triplicate and at least once for each pathogen and concentration to inform the reproducibility of the assay. Results in triplicate are also obtained from spiking into different urine samples to test for the influence of urine composition on the assay. Spiked urine is plated to blood agar plates in serial dilution to confirm the correct starting bacterial load. The algorithm to automatically call each pathogen-antibiotic combination as S or R utilizes a learning set from multiple strains of each organism. Results from later spiked samples can be used to challenge the algorithm to correctly identify AMR.

Unless defined otherwise, all technical and scientific terms used herein have the same meanings as commonly understood by one of skill in the art to which the disclosed invention belongs. Publications cited herein and the materials for which they are cited are specifically incorporated by reference. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Those skilled in the art will recognize or be able to ascertain using no more than routine experimentation, many equivalents to the specific embodiments of the invention described herein. While the invention has been described with reference to particular embodiments and implementations, it will understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### REFERENCES

1. Baltekin Ö, Boucharin A, Tano E, Andersson D I, Elf J. Point-of-care antibiotic susceptibility test. PNAS. 2017; 114(34):9170-9175.
2. Caselli, R, Ninno, A. D., Reale, R., Businaro, L., & Bisegna, P. A novel wiring scheme for standard chips enabling high-accuracy impedance cytometry. Sensors and Actuators B: Chemical. 2018. 256:580-589.
3. Chen, J.; Xue, C.; Zhao, Y.; Chen, D.; Wu, M.-H.; Wang, J. Microfluidic Impedance Flow Cytometry Enabling High-Throughput Single-Cell Electrical Property Characterization. Int. J. Mol. Sci. 2015, 16, 9804-9830.
4. Cheung K C, Di Berardino M, Schade-Kampmann G, Hebeisen M, Pierzchalski A, Bocsi J, Mittag A, Tárnok A. Microfluidic impedance-based flow cytometry. Cytometry. 2010. Part A. 77(7):648-66.
5. Coulter, W. H. High speed automatic blood cell counter and cell size analyzer. Proc Natl Electron Conf. 1956 (Vol. 12). Chicago: National Electronics Conference, Inc.; 1957; 1034-1040.
6. Kubitschek H. Electronic Counting and Sizing of Bacteria. Nature. 1958; 182:234-235.
7. Performance Standards for Antimicrobial Susceptibility Testing. 29th ed. CLSI supplement M100. Wayne, Pa.: Clinical and Laboratory Standards Institute; 2019.
8. Puttaswamy S, Gupta S K, Regunath H, Smith L P, Sengupta S. A Comprehensive Review of the Present and Future Antibiotic Susceptibility Testing (AST) Systems. Arch Clin Microbiol. 2019; 9(3):83.
9. Seydel J K, Wempe E. A simple, fast and inexpensive kinetic method to differentiate between total and viable bacteria using Coulter counter technique. Arzneimittelforschung. 1980; 30(2):298-301.
10. Schulz E, von Klitzing L, Marre R, Sack K. Relationship between the bactericidal and bacteriolytic activity of cephalosporins and changes in the cell volumes of Escherichia coli cultures. Infection. 1985; 13(5):235-9.
11. Swanton E M, Curby W A, Lind H E. Experiences with the Coulter counter in bacteriology. Appl Microbiol. 1962; 10(5):480-485.
12. Sun T, Morgan H. Single-cell microfluidic impedance cytometry: a review. Microfluidics Nanofluidics. 2010; 8(4):423-443.
13. van Belkum, A. et al., Developmental roadmap for antimicrobial susceptibility testing systems. Nat Rev Microbiol 2019; 17:51

1.-26. (canceled)

27. A system for determining the sensitivity of a target to a therapeutic formula, the system comprising;
a measurement device configured to measure properties of a target sample fluid housed within a cartridge, the cartridge having at least one test compartment and at least one control compartment, and
a processor in communication with the measurement device and a memory, wherein the processor executes computer readable instructions stored on the memory, the instructions causing the processor to perform the steps of:
measuring a measured property of the target sample fluid in the at least one test compartment under conditions favorable for target multiplication or growth,
comparing the measured property to a threshold property,
determining the sensitivity of the target to a first therapeutic formula based on a deviation of the measured property from the threshold property, and
presenting an indicator of the sensitivity of the target to the first therapeutic formula.

28. The system of claim 27, wherein the cartridge comprises two or more test compartments.

29. The system of claim 27, wherein the cartridge is less than or equal to 8 inches in width, is less than or equal to 10 inches in height, and a weight of the cartridge is less than 2 pounds.

30. (canceled)

31. (canceled)

32. (canceled)

33. The system of claim 27, wherein the cartridge does not require cold storage.

34. (canceled)

35. The system of claim 27, wherein the cartridge is fluidically sealed and houses growth media and at least one therapeutic formula.

36. The system of claim 35, wherein the system further comprises a cutting mechanism in communication with the processor, wherein the processor instructs the cutting mechanism to break a fluidic seal of the cartridge.

37. The system of claim 27, wherein the cartridge comprises a filtering chamber, the filtering chamber fluidically coupled to a target sample fluid input well and at least one test compartment.

38. The system of claim 37, wherein the filtering chamber is fluidically coupled to a mixing well, the mixing well positioned between the filtering chamber and the at least one test compartment.

39. The system of claim 27, wherein the measurement device is an impedance flow cytometry device comprising a voltage source and an amplifier.

40. (canceled)

41. The system of claim 27, wherein the at least one test compartment comprises multiple lobes bridged by at least one channel, and an electrode pair is positioned adjacent to the at least one channel.

42. The system of claim 41, wherein the test compartment is associated with a first electrode pair positioned near the top of the channel and a second electrode pair positioned near the bottom of the channel, wherein a positive electrode of the first electrode pair is adjacent to a first lateral side of the channel, and a positive electrode of the second electrode pair is adjacent to the opposite lateral side of the channel.

43. The system of claim 27, wherein the measurement device is an optical property measurement device, the optical property measurement device comprising a light source for emitting a light beam, a detector for detecting a light beam, and a path length across which the light beam travels.

44. The system of claim 43, wherein the at least one test compartment is elongate and configured to act as a light guide channel.

45. The system of claim 43, wherein the path length is from 2 centimeters to 13 centimeters.

46. The system of claim 43, further comprising at least one object that extends the path length beyond the physical distance between the light source and the detector such that the path length is greater than the distance between the light source and the detector.

47. (canceled)

48. The system of claim 27, further comprising at least one environmental control mechanism in communication with the processor.

49. The system of claim 48, wherein the measurement device and environmental control mechanisms are at least partially enclosed in a housing.

50. (canceled)

51. The system of claim 27, wherein the system is configured to receive inputs regarding the identity of the target.

52. (canceled)

53. The system of claim 27, wherein the system is configured to receive manual inputs regarding the identity of the target.

54. The system of claim 27, further comprising a fluidic and pressure handling system that is configured to couple to fluidics and pressure ports on the cartridge.

55. (canceled)

56. A system for identifying a target and determining the sensitivity of the target to a therapeutic formula, the system comprising:
an identification module configured to identify the target and quantify the target,
a target sensitivity testing module configured to communicate with the identification module, the target sensitivity testing module comprising a measurement device configured to measure properties of a target sample fluid housed within a cartridge, the cartridge having at least one test compartment and at least one control compartment, and
a processor in communication with the measurement device and a memory, wherein the processor executes computer readable instructions stored on the memory, the instructions causing the processor to perform the steps of:
measuring a measured property of the target sample fluid in the at least one test compartment under conditions favorable for target multiplication or growth,
comparing the measured property to a threshold property,
determining the sensitivity of the target to a first therapeutic formula based on a deviation of the measured property from the threshold property, and
presenting an indicator of the sensitivity of the target to the first therapeutic formula.
